(19) United States
(12) Patent Application Publication
(10) Pub. No.: US 2006/0167113 A1
(43) Pub. Date: Jul. 27, 2006

(54) Process for preparation of polymorphic form of sertraline hydrochloride

(75) Inventor: Sunil Sadanand Nadkarni, Ahmedabad (IN)

Correspondence Address: JACOBSON HOLMAN PLLC, 400 SEVENTH STREET N.W., SUITE 600, WASHINGTON, DC, 20004, US

(73) Assignee: Torrent Pharmaceuticals Ltd., Ahmedabad 380009, Gujarat State (IN)

(21) Appl. No.: 10/534,358

(22) PCT Filed: Nov. 3, 2003
(86) PCT No.: PCT/IB03/04998

§ 371 Date: May 9, 2005

(30) Foreign Application Priority Data
Nov. 7, 2002 (IN) 628/CAL/2002

(51) Int. Cl. A61K 31/135

(52) U.S. Cl. 514/657; 564/429

(57) ABSTRACT

The invention discloses a process for preparation of sertraline salts particularly sertraline hydrochloride Form V by dissolving or suspending sertraline mandelate in a solvent, reducing the pH of the solution or the suspension and isolating salt of sertraline The invention also provides for a pharmaceutical composition comprising said sertraline salt as active ingredient

### BACKGROUND OF THE INVENTION

1. Field of the Invention

This invention relates to a process for the preparation of polymorphic Form V of (1S-cis)-4-(3,4-dichlorophenyl)-1,2,3,4-tetrahydro-N-methyl-1-naphthaleneamine hydrochloride i.e. sertraline hydrochlroide. Sertraline hydrochloride is an agent for treatment for depression, obsessive-compulsive disorder and panic disorder (WO 00/32551).

2. Description of the Prior Art

The need for the drugs, which lack the obstrusive and limiting side effects of the tricyclic antidepressants had prompted the search for agents with greatly enhanced selectivity for specific mechanisms of actions believed to be essential for antidepressant efficacy. Researches targeted for selective competitive inhibitors of synaptosomal serotonin re-uptake, which led to series of 1-methylamine-4-2aryltetralins, of which the most promising was the 4-(3,4-dichlorophenyl) analogue. Testing of all possible steroisomers revealed that the required high selectivity for serotonin resides in the cis-1S,4S isomer i.e. (1S-cis)-4-(3,4-dichlorophenyl)-1,2,3,4-tetrahydro-N-methyl-1-naphthaleneamine hydrochloride (I) commonly known as sertraline hydrochloride.

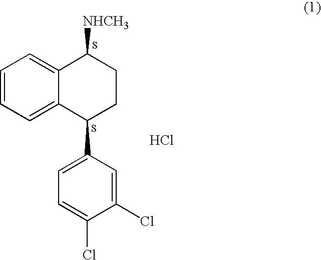

In the literature various polymorphic forms of sertraline hydrochloride have been described. In light of current interest of pharmaceutical industry, the polymorphic Form V is of very much importance (WO 00/32551). Hence, a need was felt to produce the polymorphic Form V of sertraline hydrochloride in bulk by a process which is both efficient and cost-effective.

The “sublimation-condensation method” for preparation of Form V is disclosed in U.S. Pat. No. 5,248,699. However, the said “sublimation-condensation method” is not practical on a commercial scale, considering the demand of sertraline hydrochloride Form-V. This is especially because “sublimation-condensation method” requires special assembly, wherein simultaneously high vacuum and temperature is required to be applied to sublime the starting material, whereas to collect the sublimation product, it invites the special apparatus and skills. Further more, the complexity of the issue is compounded as per the disclosure in WO 0032551, because the “sublimation-condensation method” is not found to be reproducible.

WO 0032551 and WO 0172684 mainly uses sertraline hydrochloride (Scheme-1) or sertraline base (Scheme-2) for making sertraline hydrochloride Form V.

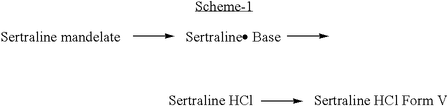

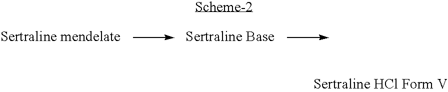

Further, WO 0132601 discloses processes for making sertraline hydrochloride Form V from using sertraline base. The preparation of sertraline hydrochloride Form V using the teachings of WO 0132601 Scheme-3 or Scheme-4. is as given below:

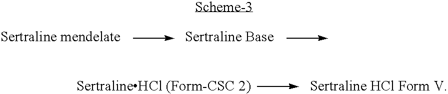

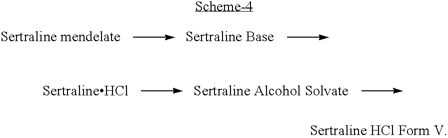

As per procedures disclosed in U.S. Pat. No. 5,248,699, U.S. Pat. No. 4,536,518, WO 032551, the sertraline base is prepared using sertraline mandelate that involves a number of steps implying increase in utilities, manpower, time required to complete the production cycle. Thus, the said processes are commercially expensive.

Thus a need was felt for production of the polymorphic Form V of sertraline hydrochloride by a simple, efficient and cost effective process.

### OBJECTS OF THE INVENTION

The first object of the present invention is to provide an efficient and cost effective process for the preparation of sertraline salts.

The second object of the present invention is to provide an efficient and cost effective process for the preparation of the polymorphic Form V of sertraline hydrochloride.

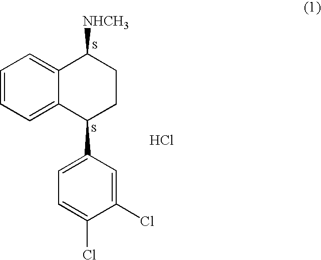

The third object of the present invention is to produce sertraline hydrochloride Form V having characteristic X-ray diffraction pattern data (XRPD).

The fourth object of the invention is to produce for sertraline hydrochloride Form V having characteristics ICR spectrum.

The fifth object of the invention is to provide a pharmaceutical composition with sertraline hydrochloride Form V as the active ingredient.

### SUMMARY OF THE INVENTION

The present invention provides for a process for the production of sertraline salt, comprising the steps of:
a) dissolving or suspending sertraline mandelate in a solvent;
b) reducing the pH of the solution or the suspension and
c) isolating salt of sertraline.

The present invention also provides for a process for the production of sertraline hydrochloride Form V comprising the steps of:
a) dissolving or suspending sertraline mandelate in a solvent;
b) reducing the pH of the solution or the suspension and
c) isolating sertraline hydrochloride Form V.

The present invention further provides for a process for preparation of a pharmaceutical composition of sertraline hydrochloride Form V by using sertraline hydrochloride Form V as active ingredient.

### DETAILED DESCRIPTION OF THE INVENTION

Sertraline hydrochloride of formula (I) exists in different polymorphic forms, viz. Form I to XVI, T1, CSC-1, CSC-2 and amorphous Form. Crystallization for polymorphs is normally done by dissolving or melting the compound followed by gradual or fast cooling of the resultant solution or molten liquid. Different polymorphic forms are identical in solution as evident from their NMR, IR (solution spectra data). On the other hand, solid-state techniques like X-ray or IR (KBr spectra) revealed the difference between polymorphic Forms.

The present invention provides new process for making sertraline hydrochloride Form V starting from sertraline mandelate.

According to the instant process, sertraline mandelate need not be converted into sertraline base and subsequently into sertraline hydrochloride unlike the prior art processes. The multiple steps involved in the prior art processes including an intermediate step for conversion of sertraline mandelate into sertraline base or sertraline hydrochloride of different Form (other than Form V) of sertraline hydrochloride is avoided because the present invention provides converting sertraline mandelate to sertraline hydrochloride Form V directly. Thus, the present invention provides the manufacturing process, which reduces number of steps implying decrease in utilities, manpower, time required to complete the production cycle. Thus, the instant invention provides a simple one-step process for production of sertraline hydrochloride Form V in an efficient and cost effective manner.

A process according to the instant invention for the production of sertraline salt, is comprising the steps of:
d) dissolving or suspending sertraline mandelate in a solvent;
e) reducing the pH of the solution or the suspension and
f) isolating salt of sertraline.

The polymorphic Form V of sertraline hydrochloride is prepared according to the instant invention by a process comprising
d) dissolving or suspending sertraline mandelate in a solvent;
e) reducing the pH of the solution or the suspension and
f) isolating sertraline hydrochloride Form V.

The solvent used for dissolving or suspending sertraline mandelate is selected from the group comprising of protic solvents or mixture thereof.

The solvent used for dissolving or suspending sertraline mandelate is selected from the group consisting of alcohol, water and mixtures thereof. The alcohols can be selected from methanol, ethanol, n-propanol, isopropanol, n-butyl alcohol, t-butyl alcohol, isobutyl alcohol and mixtures thereof. The preferable solvent is isopropanol.

The dissolving or suspending is achieved by heating and/or stirring. Heating can be done upto 90° C. Preferably sertraline mandelate is dissolved at 25-80° C. and more preferably at 25-30° C. under stirring.

Reduction of pH can be done by using organic or inorganic acids. The reduction of pH is preferably done by inorganic acids such as HCl, H₂SO₄, HNO₃.

HCl is taken in the form of gas or dissolved in a solvent. The solvent can be water or organic solvent or mixtures thereof. The organic solvent can be selected from the alcoholic solvent such as methanol, ethanol, n-propanol, isopropyl alcohol, n-butanol or mixtures thereof.

Preferably, the reduction of pH is done by using aqueous HCl.

After reduction of pH in the range of 1-3, preferably 1-2, the reaction mixture can be either clear solution or even can be kept in suspension form. The clear solution can be obtained optionally by heating upto 90° C.

The cooling is effected by allowing the solution to attain room temperature on its own or with mild coolants comprising of cold water, water, alcohols or mixtures thereof. The alcohol is selected from the group comprising of monohydroxy alcohol, dihydroxy alcohol or mixtures thereof. Further, solid obtained can be isolated to get Form V.

The process according to the instant invention is given in Scheme-5.

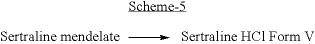

According to a preferred embodiment of the process of the instant invention, sertraline mandelate is treated with isopropyl alcoholic HCl. The pH is adjusted to 1-2 and water was added followed by heating the reaction mass to get the clear solution, which after cooling gave directly sertraline hydrochloride Form V.

The starting compound sertraline mandelate may be prepared according to the procedures disclosed in EP 30081. The preparation of highly pure sertraline mandelate is advantageous as it does not demand more time and labour for repeated crystallizations. Sertraline mandelate is prepared according to the instant invention by a process, wherein purification by repeated crystallization is not required. Also, there is no need to obtain the second crop similar to EP 30081.

A pharmaceutical composition can be obtained by using therapeutically effective amount of sertraline hydrochloride Form V thus obtained with a pharmaceutically acceptable carrier.

### BRIEF DESCRIPTION OF THE ACCOMPANYING DRAWINGS

FIG. 1 This figure indicates X-ray diffraction pattern of the compound obtained according to the present invention.

FIG. 2 This figure indicates IR spectrum of the compound obtained according to the present invention. This is a characteristic infrared absorption spectrum of the polymorphic Form V of sertraline hydrochloride of formula (I) in KBr.

The polymorphic Form V of sertraline hydrochloride of formula (I) charaterised by the following data:

Sertraline hydrochloride Form-V is characterized by powder X-ray diffraction (XRPD) pattern as set out in Table 1 given below:

TABLE 1

| | Diffraction Angle ± 0.2° | Lattice Spacing (D) |
|---|---|---|
| Serial No. | (degree two theta) | (Angstroms) |
| 1 | 5.2 | 17.119 |
| 2 | 10.9 | 8.122 |
| 3 | 14.1 | 6.259 |
| 4 | 16.3 | 5.433 |
| 5 | 17.1 | 5.181 |
| 6 | 19.0 | 4.671 |
| 7 | 19.7 | 4.506 |
| 8 | 20.9 | 4.256 |
| 9 | 22.0 | 4.046 |
| 10 | 23.0 | 3.860 |
| 11 | 23.5 | 3.776 |
| 12 | 25.3 | 3.517 |
| 13 | 25.9 | 3.437 |
| 14 | 29.0 | 3.075 |

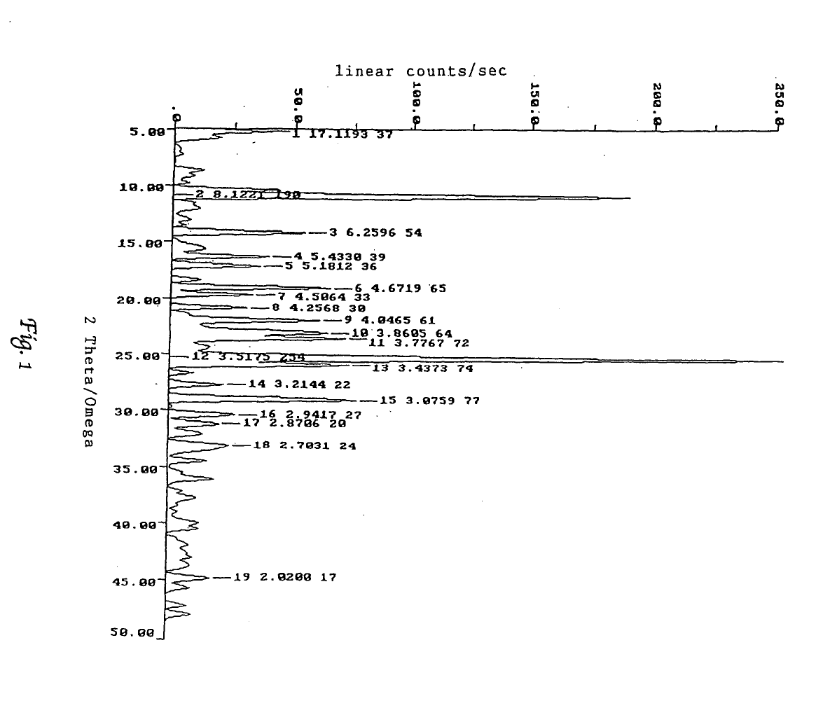

The sertraline hydrochloride that results from practicing the invention as exemplified herein can be characterised by its powder X-ray diffraction pattern. FIG. 1 is a representative pattern of sertraline hydrochloride Form V. The principal peaks observed are at about 5.2±0.2, 10.9±0.2, 14.1±0.2, 16.3±0.2, 17.1±0.2, 19.0±0.2, 19.7±0.2, 20.9±0.2, 22.0±0.2, 23.0±0.2, 23.5±0.2, 25.3±0.2, 25.9±0.2 and 29.0±0.2 02 theta.

The IR spectrum of sertraline hydrochloride Form V produced by present process is characterized by the following bands:

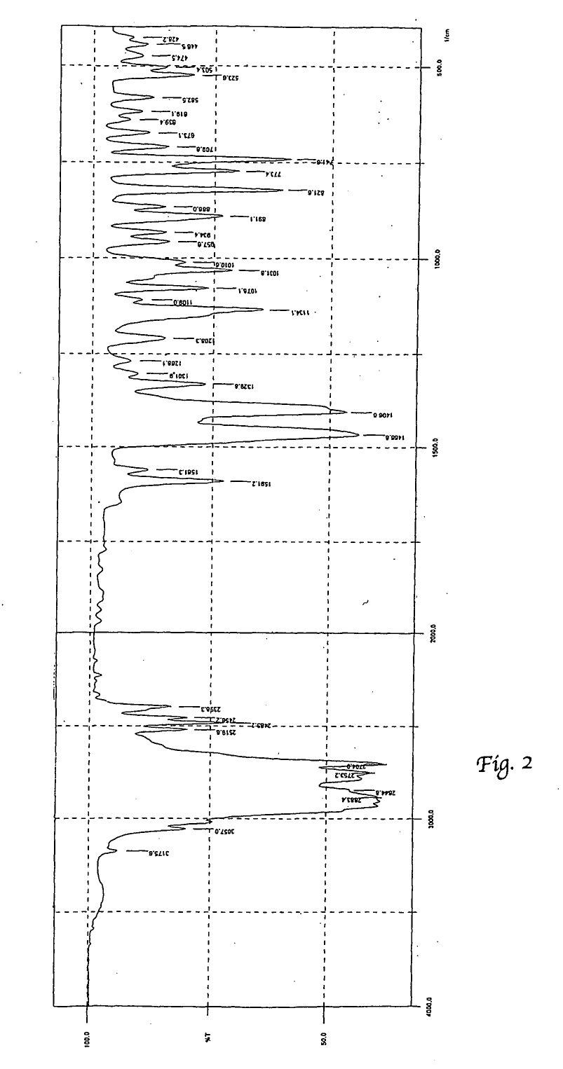

773 cm⁻¹, 1011 cm⁻¹, 1032 cm⁻¹, 1054 cm⁻¹, 1134 cm⁻¹, 1330 cm⁻¹, 1561 cm⁻¹ and 1591 cm⁻¹ as shown in FIG. 2.

FT IR spectrum was recorded in solid state as KBr dispension using Shimadzu FT IR 8700 series FT IR Spectrophotometer.

The pharmaceutical composition of sertraline hydrochloride Form V can be prepared by using the above referred chemical compound complying the following tests:

| Sr. | | |
|---|---|---|
| No. | Tests | Limits |
| 1. | Related substances (%) | Not more than 0.50 |
| | (by HPLC) | |
| | Total known and unknown | |
| | impurities | |
| 2. | Sulphated ash (%) | Not more than 0.2 |
| 3.. | Heavy Metals (ppm) | Not more than 20 |
| 4. | Assay (%)(By titration) | 98.0 to 102.0; on anhydrous |
| | | basis |
| 5. | Residual solvents (ppm) | |
| | (a) Isopropyl alcohol | Not more than 2000 |
| | (b) Methanol | Not more than 100 |
| | (c) Acetone | Not more than 100 |
| | (d) Methylene chloride | Not more than 200 |
| 6. | Polymorph by XRD | 2 Theta Values(D): |
| | | 5.2(17.119), 10.9 (8.122), |
| | | 14.1(6.259), 16.3 (5.433), |
| | | 17.1(5.181), 19.0 (4.671), |
| | | 19.7(4.506), 20.9 (4.256), 22.0 |
| | | (4.046), 23.0 (3.860), 23.5 |
| | | (3.776), 25.3 (3.517), 25.9 |
| | | (3.437) and 29.0 (3.075) |
| 7. | IR (cm⁻¹) | 773, 1011, 1032, 1054, |
| | | 1134, 1330, 1561 and 1591 |
| 8. | Particle size (By Sizer) | Not less than 90.0% |
| | Below 20 μm | |
| 9. | Microbial limit tests | |
| | Total aerobic count (cfu/g) | Not more than 1000 |
| | Total fungal count (cfu/g) | Not more than 100 |
| | E. coli | Should be absent |

In the following section preferred embodiments are described by way of examples to illustrate the process of this invention. However, this is not intended in any way to limit the scope of the present invention.

### PREPARATORY EXAMPLES

Preparation of Sertraline Mandelate from Racemic HCl Salt of Sertraline.

In a one liter round bottom flask methylene chloride (250 ml), water (250 ml) and racemic HCl salt of sertraline (50 gm) at room temperature were taken. To it 20% sodium hydroxide solution (10 gm sodium hydroxide solution in 50 ml of water) was added to adjust pH between 9 to 10 as detected on pH paper. Stirred for 45 minutes till clear solution was obtained. Methylene chloride layer was separated and aqueous layer extracted with methylene chloride twice (50 ml for each extraction). All methylene chloride layers combined and washed with water till the pH reaches at 7 to 8. All methylene chloride layers are collected and distilled out under vacuum at 60° C. to get an oil. Methanol 200 ml is charged into it and then heated to 50-55° C. D(−) Mandelic Acid solution (23 gm in 50 ml methanol) added to it at 55-60° C. The temperature raised to 60-65° C. and maintained for 10 minutes. The mass cooled to 30-35° C. in 1 hr. and further chilled to 20-25° C. and temperature maintain at that level for 30 minutes to get solid. The solid is filtered and washed with acetone 3 times (25 ml each) to get sertraline mandelate with dry weight: 28.0 gm.

Preparation of Sertraline Hydrochloride Form V from Sertraline Mandelate

In 1 litre 4 neck round bottom flask equipped with stirrer, theremometer pocket and water condenser, sertraline mandelate (25 gm) was added at room temperature. To it, 200 ml of isopropyl alcohol was added under stirring. The pH of the solution was adjusted to 1 to 2 by adding concentrated HCl. To it, 5 ml water was added and heated to reflux to get the clear solution. The solution was filtered through hyflow bed and cooled it to room temperature to get 23 gm of the white solid which was dried further to get 13 gm of dried material of Form V.

Pharmaceutical Compositions

The pharmaceutical compositions of sertraline hydrochloride Form V should preferably have a particle size below 20μ and purity not less than 90% when prepared in admixture with pharmaceutically acceptable diluent, carrier or excepient. The impurity level of sertraline hydrochloride in such composition should preferably not exceed 0.50% with sulphated ash content not more than 0.2% and heavy metals not more than 20 ppm preferably sertraline hydrochloride used for such composition has the assay figure by titration between 98.0 to 102% on anhydrous basis.

The residual solvents in such composition are preferably in the following limits:

| | |
|---|---|
| (a) isopropyl alcohol | not more than 2000 ppm |
| (b) methanol | not more than 100 ppm |
| (c) acetone | not more than 100 ppm |
| (d) methylene chloride | not more than 200 ppm |

The microbial limits in such composition are preferably as under:

| | |
|---|---|
| total aerobic count (cfu/g) | not more than 1000 |
| total fungal count (cfu/g) | not more than 100 |
| E. Coli | should be absent. |

While the present invention has been described in terms of its specific embodiments, certain modifications and equivalents will be apparent to these skilled in the art and are intended to be included within the scope of the present invention.

1. A process for the production of sertraline hydrochloride Form-V comprising the steps of:
a) dissolving or suspending sertraline mandelate in a protic solvent or a mixture of protic solvents;
b) reducing the pH of the solution or the suspension by adding hydrochloric acid in water to form a clear solution; and
c) isolating sertraline hydrochloride Form V.

2. The process as claimed in claim 1, wherein protic solvent(s) used in step (a) is selected from the group comprising of alcohol, water or mixtures thereof.

3. The process as claimed in claim 2, wherein said alcoholic solvent used in step (a) is selected from the group comprising of methanol, ethanol, n-propyl alcohol, isopropyl alcohol, n-butyl alcohol, t-butyl alcohol and isobutyl alcohol or a mixture thereof.

4. The process as claimed in claim 3, wherein said alcoholic solvent is isopropyl alcohol.

5. The process as claimed in claim 1, wherein said step(a) of dissolving or suspending is achieved by heating and/or stirring.

6. The process as claimed in claim 1, wherein said step (a) of dissolving or suspending sertraline mandelate in a solvent is carried out at temperature in the range of 20 to 90° C.

7. The process as claimed in claim 6, wherein said range of temperature is 25 to 80° C.

8. The process as claimed in claim 7, wherein said range of temperature is 25 to 30° C.

9. The process as claimed in claim 1, wherein pH is reduced to the range of 1 to 3 in step (b).

10. The process as claimed in claim 9, wherein pH is reduced to the range of 1 to 2.

11. The process as claimed in claim 1, wherein isolation of sertraline hydrochloride Form V in step (c) is carried out by cooling the contents of step (b).

12. The process as claimed in claim 11, wherein the cooling is effected by allowing the solution to attain room temperature on its own or with mild coolants comprising of cold water, water, alcohol or mixtures thereof.

13. The process as claimed in claim 12, wherein said alcohol is selected from the group comprising of monohydroxy alcohols, dihydroxy alcohols or mixtures thereof.

14. A process for preparation of an immediate release pharmaceutical composition of sertraline hydrochloride Form-V, comprising mixing sertraline hydrochloride Form-V, of particle size below 20μ is not less than 90% with pharmaceutically acceptable diluent, carrier or excepient.

15. The process for preparation of a pharmaceutical composition as claimed in claim 14, wherein the impurity level in sertraline hydrochloride Form V used is not more than 0.50% comprising of both known and unknown impurities.

16. The process for preparation of a pharmaceutical composition as claimed in claim 15, wherein the sulphated ash in sertraline hydrochloride Form V is not more than 0.2%.

17. The process for preparation of a pharmaceutical composition as claimed in claim 15, wherein the heavy metals in sertraline hydrochloride Form V used is not more than 20 ppm.

18. The process for preparation of a pharmaceutical composition as claimed in claim 14, wherein the assay by titration of sertraline hydrochloride Form V is between 98.0 to 102.0% on anhydrous basis.

19. The process for preparation of a pharmaceutical composition of as claimed in claim 14, wherein the residual solvents in the active ingredient sertraline hydrochloride Form V are:

| | |
|---|---|
| (a) isopropyl alcohol | not more than 2000 ppm |
| (b) methanol | not more than 100 ppm |
| (c) acetone | not more than 100 ppm |
| (d) methylene chloride | not more than 200 ppm |

20. The process for preparation of a pharmaceutical composition as claimed in claim 14, wherein the microbial limits in active ingredient sertraline hydrochloride Form V are:

| | |
|---|---|
| total aerobic count (cfu/g) | not more than 1000 |
| total fungal count (cfu/g) | not more than 100 |
| E. Coli | should be absent. |

21. A process for the preparation of sertraline hydrochloride Form-V, substantially as herein described, particularly with reference to the foregoing examples.
